(19) United States
(12) Patent Application Publication
(10) Pub. No.: US 2006/0205051 A1
(43) Pub. Date: Sep. 14, 2006

(54) Process for anaerobic oxidation of methane

(76) Inventors: Alfons Johannes Maria Stams, Wageningen (NL); Cees Jan Nico Buisman, Harich (NL); Hendrik Dijkman, Ijlst (NL); Willem Meindert De Vos, Bennekom (NL)

Correspondence Address: GREENBERG TRAURIG LLP, 2450 COLORADO AVENUE, SUITE 400E, SANTA MONICA, CA, 90404, US

(21) Appl. No.: 10/535,750

(22) PCT Filed: Nov. 20, 2003
(86) PCT No.: PCT/NL03/00818

§ 371 Date: Dec. 19, 2005

(51) Int. Cl. C12P 3/00; C12N 1/20

(52) U.S. Cl. 435/168; 435/252.3

(57) ABSTRACT

Methane can be used as a source for producing hydrogen or hydrogen equivalents in a biological process using mesophilic or thermophilic bacteria of the order of the Thermotogales, e.g. Thermotoga maritima. Hydrogen can be produced as such, or as a reducing equivalent for the reduction of various compounds such as sulphur compounds. Especially, the methane oxidation can be used for anaerobically reducing sulphate to sulphide using methane as the predominant or sole electron donor, in coculture with sulphate-reducing micro-organisms.

### FIELD OF THE INVENTION

The present invention relates to an oxygen-free biological process for converting methane to hydrogen or hydrogen equivalents. Furthermore, the invention relates to a biological process of reducing sulphur compounds to sulphide.

### BACKGROUND

Biological methane oxidation in the presence of oxygen is a well-established process in natural habitats and in industrial applications. It is reported that aggregates of archaea and sulphate-reducing bacteria are capable of methane oxidation in deep-sea conditions (Boetins et al, Nature, 407: 623-626 (2000), Hoehler et al, Global Biogeochemical Cycles, 8: 451-563 (1994). However, no pure or defined microbial cultures are known that are capable of anaerobic methane oxidation (Orphan et al., Proc. Nat. Acad. Sci. USA (2002), 99, 7663-7668). Hence, anaerobic oxidation of methane is not well-understood and is not applied on an industrial scale. The use of archaea in an industrial process is hardly feasible, if at all, because of their extremely low growth rates. (?)

Removal of sulphur oxide compounds such as sulphate, sulphite, sulphur dioxide, thiosulphate and the like and elemental sulphur by anaerobic conversion to sulphide at moderate or high temperature is well known, e.g. from EP-A-0451922, WO 92/17410, WO 93/24416 and WO 98/02524. These processes usually require an electron donor (or hydrogen donor) which can be hydrogen, carbon monoxide or organic molecules such as alcohols and fatty acids.

It was found recently (Balk et al., Int. J. Syst. Evol. Microbiol. (2002), 52, 1361-1368) that certain Thermotoga species are capable of anaerobically degrading methanol, alone, in coculture with Methanothermobacter or Thermodesulfovibrio species, or in the presence of sulphur, sulphur oxide compounds or organic sulphur compounds, such as anthraquinone-2,6-disulphonate.

### SUMMARY OF THE INVENTION

It was found according to the invention that anaerobic thermophilic bacteria of the order of the Thermotogales, especially from the species Thermotoga, are capable of converting methane, in the absence of oxygen, to hydrogen or hydrogen equivalents. The methane carbon atom was found to be converted to carbon dioxide and was not incorporated in the biomass. The hydrogen produced can be used as such, or can be used to provide hydrogen equivalents suitable for reducing various compounds e.g. sulphur compounds such as sulphate, sulphate and thiosulphate. It was furthermore found that hydrogen equivalents required for biological reduction reactions can be effectively provided by methane oxidation by anaerobic methane-oxidising bacteria. Thus the invention concerns a process of producing hydrogen or hydrogen equivalents by anaerobically subjecting methane to the activity of one or more Thermotogales species. Similarly, the invention concerns a process of anaerobic oxidising methane using a Thermotogales species or strain. Furthermore, the invention concerns a process for biological reduction of chemicals such as sulphur compounds and metals, wherein the required hydrogen equivalents are produced by subjecting methane to anaerobic methane-oxidising bacteria.

### DESCRIPTION OF THE INVENTION

The invention pertains to the anaerobic, bacterial production of hydrogen or hydrogen equivalents. In the present context, hydrogen equivalents are understood to comprise atoms, molecules or electrons, i.e. reduction equivalents, which lower the oxidation state of a substrate. These include e.g. acetate and formate. They are also referred to as electron donors. Where the present process produces hydrogen equivalents, as distinct from (molecular) hydrogen, the process is carried out in the presence of a suitable substrate capable of accepting the hydrogen equivalents. Where reference is made to methane, also higher alkanes and alkenes, such as ethane, ethene, propane, etc. are contemplated.

The bacteria to be used according to the invention are anaerobic methane-oxidising (alkane-oxidising) bacteria. These bacteria include terrestrial and aquatic (marine) species, which can be obtained from hydrothermal sources, oil-wells, and sometimes anaerobic thermophilic bioreactors. They can grow under a variety of environmental conditions and, depending on the natural source and possibly adaptation processes, they can be mesophilic and/or thermophilic. Examples of suitable bacteria belong to the order of the Thermotogales, which are mostly thermophilic. A description thereof is given by Wery et al. (FEMS Microbiol. Biol. 41, (2002) 105-114) and Reysenbach et al. (Int. J. Syst. Evol. Microbiol., 52, (2002) 685-690). The use in producing hydrogen from organic sources such as sugars is described in WO 02/06503. The Thermotogales include the genera Marinitoga, Geotoga, Petrotoga, Thermotoga, Thermosipho and Fervidobacterium. They are especially from the group containing the latter three genera. These include the species (with DSM accession numbers) Thermotoga Retina (DSM 3109), Thermotoga thermarum (DSM 5069), Thermotoga hypogea (DSM 11164), Thermotoga subterranea (DSM 9912), Thermotoga elfei (DSM 9442), Thermotoga lettingae (DSM 14385), Thermosipho melanesienis (DSM 12029), Thermosipho geolei DSM 13256), Fervidobacterium islandicum (DSM 5733) and F. nodosum (DSM 5306). Most of them are available in recognised culture collection such as DSM or ATCC, and the genome of some of them, such as Thermotoga maritima, has been sequenced (Nelson et al., Nature (1999), 399, 323-329).

The methane-oxidising bacteria may be used as a pure culture of one of the species or strains mentioned above or as a defined mixture with other bacteria, or as a part of a mixed culture obtained from environmental samples or from bioreactors, if necessary and preferably after adaptation to the desired process conditions. The use of a pure culture has the advantage of allowing the process to be controlled as desired. The invention also concerns such pure cultures as well as defined combinations of cultures as further illustrated below.

The species to be used in the process of the invention are mesophilic or thermophilic species. The thermophilic species have their maximum activity between 50 and 100° C., but they are generally sufficiently active in the mesophilic temperature range for the process to be cared out at temperatures between 30 and 50° C. as well, or even from 25° C. upwards, if necessary after adaptation. Mesophilic species have their maximum activity between 30 and 50° C., but axe sufficiently active from 20° C. and up to e.g. 60° C. The most preferred temperatures for the process of the invention are between 25 and 90° C., most preferably between 30 and 60° C.

In an embodiment of the process of the invention, the anaerobic methane oxidation is performed for producing molecular hydrogen. The relevant total reaction can be simplified as follows:
CH₄+2H₂O→4H₂+CO₂
The culture medium contains basic mineral medium supplemented with growth factors into which methane is introduced e.g. by sparging or another method tat ensures intimate contact with the micro-organisms. The hydrogen produced can be collected e.g. using gas recirculation, wherein the gas is contacted with a selective membrane which is permeable for hydrogen and impermeable for larger molecules including methane, and the remaining gas can be recirculated to the methane-oxidising reactor. Alternatively, suitable selective absorbents can be arranged in such a manner that the gas evolving from the reactor is contacted with the absorbents. Efficient withdrawal of hydrogen from the reaction mixture ensures sufficient bioconversion of methane to hydrogen. The hydrogen produced can be used as a fuel or as a chemical synthesis agent or in biological or chemical reduction processes.

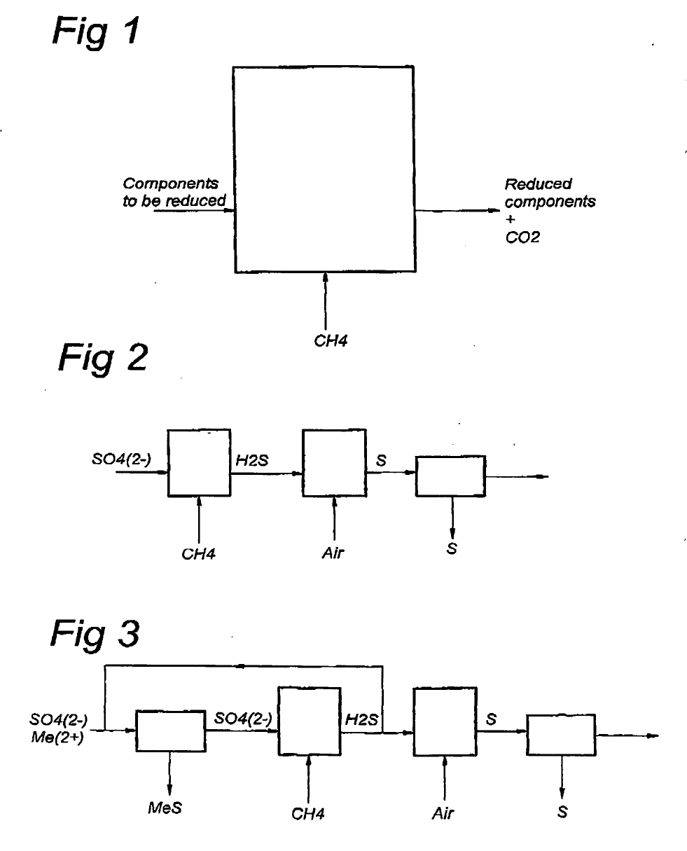

In a preferred embodiment, the anaerobic methane oxidation is performed for reducing substrates, such as nitrate, azo compounds, inorganic and organic sulphur compounds such as elemental sulphur, sulphate, sulphite, thiosulphate, polysulphides, anthraquinone-2,6-disulphonate, dissolved metals, oxidised halogen compounds, nitrate and other compounds a must be removed. The compounds can be present in liquid waste streams, if appropriate after extraction from the gas stream by scrubbing or the like. They can also be present e.g. as soil contaminants. The compounds to be reduced can also be present in production lines, for producing desired reduced compounds. The following reaction may apply:
Ca₄+4A+2H₂O→4AH₂+CO₂
wherein A is a hydrogen acceptor, and AH₂ may be replaced by equivalents or subsequent conversion products. According to this embodiment, methane-oxidising bacteria to be used include those of the Thermotogales order as described above, as well as other methane-oxidisers, e.g. those related to Desulfosarcina. The reduction step itself is in particular a biological reduction using suitable respiring organs. This process is schematically illustrated in FIG. 1.

In a particularly preferred embodiment, the anaerobic methane oxidation is used to reduce oxidised sulphur compounds, such as sulphate, sulphite and elemental sulphur. In the following, reference is made to sulphate, but other sulphur-oxygen species, such as sulphite and hydrogenated (e.g. bisulphite) and neutral (e.g. sulphur trioxide) equivalents are also comprised. The relevant total reaction can be simplified as follows:
CH₄+SO₄²⁻→HCO₃⁻+HS⁻+H₂O
This embodiment requires the presence of agents capable of transferring hydrogen equivalents to sulphate. Such agents are especially sulphate-reducing micro-organisms, which are known in the art. Suitable sulphate-reducing micro-organisms include mesophilic and thermophilic hydrogen-utilising stains from the bacterial sulphate-reducing genera, e.g. Desulforomonas, Desulfovibrio, Thermodesulfovibrio and Desulfotomaculum (e.g. the strain described in WO 98/02524) as well as the archaeal sulphate-reducing genus, e.g. Archaeoglobus, such as A. profundus.

The conversion of sulphate by a coculture comprising the anaerobic methane oxidisers as described above can be carried out in a conventional bioreactor having an inlet for sulphate-containing water, e.g. originating from a gas desulphurisation plant, a gas inlet for methane supply, a liquid outlet for sulphide-containing water, a gas outlet for the resulting gas mixture containing e.g. residual methane, hydrogen, hydrogen sulphide, and optionally means for supporting the biomass and for keeping it in effective contact with the liquid and (dissolved) gaseous materials, optional filters for separating gaseous products from the culture mixture and means for maintaining the desired reactor temperature. Furthermore, a gas separation unit may be provided for separating the resulting gas mixture and returning recovered methane as well as a treatment unit for treating hydrogen sulphide, e.g. a unit for biologically converting sulphide to elemental sulphur and for separating off the sulphur. Since the bacteria do not use methane for their cell synthesis, further carbon sources, e.g. methanol, ethanol, organic acids, yeast extract or components thereof, or other organic matter should be supplied to the bioreactor, in addition to methane.

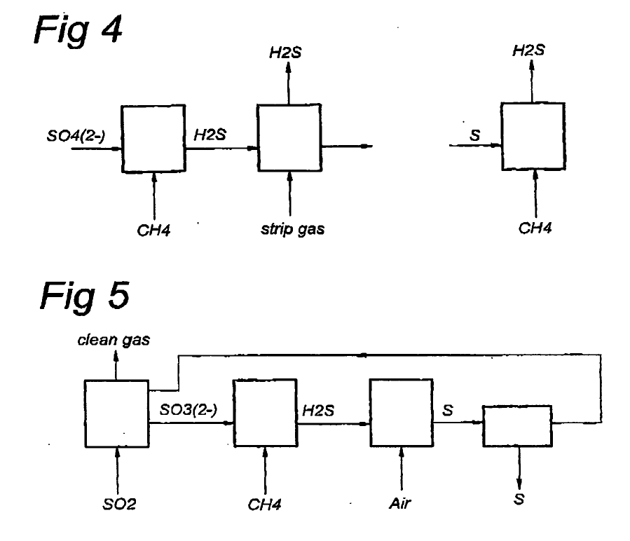

Variations in the process of reducing sulphur compounds using a coculture of methane-oxidisers and sulphate-reducers are schematically illustrated in FIGS. 2-5. FIG. 2 is a flow diagram for reducing sulphate to sulphide, followed by biological oxidation of sulphide to elemental sulphur. FIG. 3 shows sulphate reduction in combination with metal precipitation in the form of metal sulphides (MeS) by the hydrogen sulphide produced and oxidation of the surplus hydrogen sulphide to elemental sulphur. FIG. 4 shows two variants of a process for producing hydrogen sulphide, either by separate stripping, or by hydrogen sulphide removal using the methane stream. The hydrogen sulphide can be concentrated and used for sulphuric acid production. FIG. 5 illustrates sulphur dioxide removal from gases by scrubbing (first stage) followed by biological reduction as in FIG. 2.

In another embodiment of the process of the invention can be used for reducing noxious bromate or chlorate to less noxious bromide and chloride. These compounds can be present in process water from chemical industries. The reduction of bromate or chlorate requires the presence of bromate- or chlorate reducing species, which can be sulphate-reducing bacteria and archaea. Species capable of reducing chlorate or bromate those of the genera Dechlorosoma, Dechloromonas and Pseudomonas such as Pseudomonas chloritidismutans, Dechloromonas agitata, Dechlorosoma suillum, strain GR-1. Similarly, nitrate reduction can be performed using commonly known denitrifiers. Known denitrifying bacteria include Pseudomonas stustzeri, Paracoccus denitrificans, Haloarcula marismortui and Staphylococcus aureus.

According to a further embodiment, the process may be used for reducing metal ions to their low-valence or zero-valence state. They can be precipitated and separated in these lower valence states e.g. as oxides, hydroxides, carbonates, phosphates, sulphides or neutral metals. The biological reduction of metals is described for example in WO 00/39035. Examples of metals that can be reduced and converted to insoluble metals or insoluble metal oxides, hydroxides or the like include selenium, tellurium, uranium, molybdenum, vanadium, chromium, and manganese. Bacteria capable of reducing these metals include the genera Geobacter, Pseudomonas, Shewanella, Desulfovibro, Desulfobacterium, Desulfomicrobium, Desulforomonas and Alteromonas. If desired, a moving sand filter can be used for separating the resulting metal precipitates as described in WO 00/39035.

The use of the methane-oxidising bacteria in providing reducing equivalents in (biological) reduction processes is beneficial in technical and economical terms. Current process using methane as the ultimate reducing agent require the intermediary use hydrogen to be produced from methane by chemical (catalytic) reforming. This implies the investment in and use of reformers or similar equipment and also consumes about 50% of the methane by combustion needed to keep the catalytic process at the necessary high temperature. These drawbacks are eliminated by the present biological process, thus resulting in substantial cost savings both in equipment cost, and in operational cost (e.g. 50% lower methane consumption).

The process of the invention can be carried out in a conventional bioreactor of the anaerobic type, having means for introducing a gas into the reactor contents and means for carrying off gases from the headspace of the reactor. The reactor can be of the stirred type, but preferably the reactor is of a type having biofilms, present on carrier particles such as sand, basalt, polymer particles etc., or in the form of granules, plates, membranes and the like, in order to optimise contact between the substrate (methane) and the micro-organisms, and—in case of coculture—between the different microorganisms.

An example of a suitable reactor type for the biological conversion is the so-called gaslift-loop reactor. This is a type of reactor which is especially beneficial when a gaseous substrate has to be supplied for the reaction. It is operated using a vertical circulation activated by the gas (methane) introduced at the bottom of the reactor. An example of such a reactor is the 500 m³ gaslift-loop reactor used at the zinc plant of Budel Zinc in the Netherlands. In this case 10 tons/day of sulphate is reduced biologically by addition of 12,000 nm³/day of hydrogen gas. Additionally, part of the bioreactor off-gas is recycled by compressors to improve the efficiency of the hydrogen utilisation Here daily 20000 nm³ of natural gas is converted in a steam reforming unit in order to provide the required amount of hydrogen gas. When using anaerobic methane oxidation as described above, the reformer a be by-passed and methane can be introduced in the reactor directly.

Another suitable reactor type is a membrane bioreactor, wherein biomass retention is effected by passing the reactor effluent through a (membrane) filter. A membrane bioreactor can also be useful for separating a gas product (such as hydrogen or hydrogen sulphide). In such as embodiment, a membrane which is permeable for the gas (e.g. hydrogen sulphide) separates reactor liquid from the gas space of the reactor, where an absorbing gas is passed for carrying away the gas. This allows low hydrogen sulphide levels to be maintained in the reactor without requiring a high water flow through the reactor.

The process of the invention can be carried out at atmospheric pressure, or—if desired—at elevated pressure, e.g. pressure in the of 10-100 bar, using appropriate pressure-resistant equipment. Elevated pressures may have the advantage of increasing the conversion rate of the biological processes using methane.

### EXAMPLES

Strains. Thermotoga maritima (DSM 3109) and Archaeoglobus profundus (DSM 5631) were purchased from the Deutsche Sammlung von Mikroorganismen und Zellkulturen (Braunschweig, DE). T. lettingae (DSM 14385) and Desulfotomaculum sp. strain WW1 were isolated in our laboratory.

Cultivation techniques. Anaerobic culture techniques were used throughout this study. The cells were grown in an anaerobic medium typically supplemented with 0.15 g/l yeast extract. The medium contained (per liter demineralised water) 0.335 g of KCl, 4.0 g of MgCl₂. 6H₂O, 3.45 g of MgSO₄. 7H₂O, 0.25 g of NH₄Cl, 10 g of NaCl, 0.10 g of K₂PO₄, 4.0 g of NaRCO₃, 0.5 g of Na₂S. 9H₂O, 1.0 g of Na₂SO₄, 0.73 g of CaCl₂.2H₂O, 10 ml of trace element and 5 ml of two-times concentrated vitamin solution which were based on medium 141 of DSM (http://www.dzmz.de). The medium was boiled and cooled to room temperature under a stream of O₂-free N₂ gas. The medium was anaerobically dispensed into serum bottles and a gas phase of 180 kPa N₂/CO₂ (80/20, v/v) was applied. The bottles were closed with butyl rubber stoppers and sealed with crimp seals. The medium was autoclaved for 20 min at 121° C. Stock solutions of NaHCO₃, Na₂S, CaCl₂ and vitamin solutions wee prepared under a nitrogen gas atmosphere and added after sterilisation. The vitamin solution contained higher amounts of vitamin-B₁₂ and vitamin-B₁ resulting in concentrations in the medium of 0.5 and 0.1 mg. 1⁻¹, respectively. Thiosulfate was added from 1 M filter-sterilized stock solution. Calculated amounts of ¹³C-methane were injected into bottles prior to autoclaving. For coculture experiments, A. profundus and Desulfotomaculum sp. strain WW1 were grown on H₂ and CO₂ in 248 ml serum vials with 50 ml medium at 80° C. and 65° C., respectively. As additional carbon source, 1 mM of sodium acetate was added. The sulphate reducers were grown for 1 day and then the gas phase was changed to 180 kPa N₂/CO₂ (80/20, v/v) and CH₄ gas was added to the final concentration of 1.75 mmol per vial. The A. profundus culture was inoculated with T. maritima and the Desulfotomaculum sp. with T. lettingae. For inoculation, methane adapted cultures of T. maritima and T. lettingae were used. Since the medium already contained nearly 1 mmol of sulphate per vial, no sulphate was added.

Analytical techniques. Highly pure (min 99% ¹³C) methane gas was obtained from Campro Scientific B.V. (Veenendaal, NL). Serum vials were prepared with approximately 1.75 mmol of ¹³C-methane as substrate in 50 ml medium. Control bottles were prepared with or without ¹³C-methane, thiosulphate and organisms. NMR-tubes contained the sample, 10% (v/v) D₂O and 100 mM dioxane to give a final volume of 15 ml. The proton-decoupled ¹³C-NMR-spectra of the samples were recorded at 75.47 MHz on a Bruker AMX-300 NMR spectrometer. For each spectrum 7200 transients (5 h) were accumulated and stored on disc using 32 k data points, a 45° pulse angle and a delay time of 1 s between pulses. The measuring temperature was maintained at 10° C. and the chemical shift belonging to the dioxane carbon nuclei (67.4 ppm) was used as an internal standard. The deuterium in the samples (10%, v/v) was used for field lock and dioxane as an internal standard.

Hydrogen and methane were determined at room temperature by either gas chromatography (GC) (see: Stams et al. Appl. Environ. Microbiol. (1993) 59 114-1119) or GC (Hewlett Packard model 5890) equipped with a mass selective detector (MS). Methane, carbon dioxide and their stable isotopes were separated on a capillary column (innowax, 30 m×0.25 mm (df=0.5 μm), Packard, NL) with helium as the carrier gas. Gas samples (200 μl) were injected in a split injector (inlet pressure 1 kPa; split ratio 25:1) at a column temperate of 35° C. Methane and carbon dioxide and their stable isotopes were monitored at m/z 16 and 17, and 44 and 45, respectively. Total methane and CO₂ concentrations were determined quantitatively by gas chromatography (see Stams et al. (1993 above). The H¹³CO₃⁻ concentration in the liquid phase was calculated from the amount of CO₂ which accumulated in the gas phase after acidification. Thiosulphate and sulphate were analysed by HPLC (see: Scholten and Stams, Antonie van Leeuwenhoek (1995) 68, 309-315). Sulphide was determined as described by Trüper and Schlegel (see: Antonie van Leeuwenhoek (1964) 30, 225-238).

### Example 1

### Anaerobic Methane Oxidation by Thermotoga Species

Thermotoga maritima and T. lettingae were incubated with highly pure ¹³C-labelled methane under strictly anaerobic conditions in the presence of thiosulphate as the electron acceptor. The experiments were carried out was studied in duplicate cultures at incubation temperatures of 80° C. and 65° C., respectively. Methane oxidation and product formation were determined by analysing the gas phase by gas chromatography (GC) and GC-mass spectroscopy (MS) and by using Nuclear Magnetic Resonance spectroscopy (NMR) for liquid samples.

In acidified samples, the total CO₂ which accumulated in the gas phase after 40 days of incubation was measured by using GC (Table 1). In the control incubation without methane which contained totally 4.87 mmol of CO₂ per vial, slight growth was observed due to the presence of yeast extract in the medium and consequently, unlabelled CO₂ was found. Growth in the presence of ¹³C-methane and thiosulphate by T. maritima and T. lettingae resulted in significantly increased cell numbers (Table 1). The stoichiometry of methane conversion by the two cultures yielded nearly equal amounts of products after 40 days of incubation. The percentage of ¹³C in CO₂ was 5.9% in liquid phase and 2.1% in gas phase for T. lettingae. These values for T. maritima were 6.2% and 2.4%, respectively. The ratio of methane oxidation to carbon dioxide and thiosulphate reduction to sulphide in both samples were approximately 1:1 and 1:2, respectively. Nearly 1 mmol of ¹³C-metane per vial was utilised by both bacteria. The rest of the methane in the vials was not utilised even after 40 days of prolonged incubations. However, when less methane was added (up to 0.5 mmol per vial), all ¹³C-methane was completely utilised by the two bacteria. In both cases, measurable methane conversion started after around 10 days. The rates of anaerobic methane transformation by T. maritima and T. lettingae were 32 and 30 μmol per vial per day, respectively. The ¹³C-carbon recoveries for T. maritima and T. lettingae were calculated to be 82% and 79%, respectively.

TABLE 1 a ¹³C-methane oxidation in the presence of
thiosulphate by T. maritima‡.

| | | | | Total | | Number of |
|---|---|---|---|---|---|---|
| | | Thio- | Total | Sulphide | Hydro- | cells |
| | ¹³CH₄ | sulphate | CO₂* | # | gen | (ml)† |
| Day 0 | 1.75 | 0.99 | 4.87 | 0.05 | 0.00 | 4 × 10⁵ |
| Day 40 | 0.68 | 0.00 | 5.75 | 1.96 | 0.05 | 8 × 10⁷ |
| Utilised/ | 1.07 | 0.99 | 0.88 | 1.91 | 0.05 | |
| produced | | | | | | |

TABLE 1 b ¹³C-methane oxidation in the presence of
thiosulphate by T. lettingae‡.

| | | | | Total | | Number of |
|---|---|---|---|---|---|---|
| | | Thio- | Total | Sulphide | Hydro- | cells |
| | ¹³CH₄ | sulphate | CO₂* | # | gen | (ml)† |
| Day 0 | 1.75 | 1.01 | 4.87 | 0.05 | 0.00 | 5 × 10⁵ |
| Day 40 | 0.71 | 0.00 | 5.68 | 1.39 | 0.04 | 1 × 10⁷ |
| Utilized/ | 1.05 | 1.01 | 0.81 | 1.84 | 0.04 | |
| produced | | | | | | |

‡The values were calculated in mmol per vial and corrected with the control samples. All measurements were done in duplicates and the highest values of each sample were calculated
*Total CO₂ includes CO₂ from the medium composition in liquid and gas phases and ¹³C-carbon dioxide formed
#Total sulphide includes sulphide from the medium composition in liquid and gas phases and sulphide formed during methane oxidation
†Ballooning cells were not taken into account during counting of the cells

### Example 2

### Biomass Analysis

Thermotoga lettingae and Thermotoga martima were grown with labelled methane and thiosulfate at 65 and 80° C., respectively. After growth, cells were centrifuged. The percentage ¹³C in the supernatant and the cell pellet were analysed. As shown before, ¹³C labelled bicarbonate was formed, but we could not detect any label incorporation in biomass. This indicates that no cell biomass is formed from methane or its degradation products, but from yeast extract supplied to the medium. Thus, it seems that the bacteria have a split metabolism.

Enzyme activity: Methane-oxidizing activity was determined in cell free extracts prepared from methane grown cells. We could measure high activities of an NAD-dependent methane dehydrogenase (>1 □mol.min⁻¹.mg⁻¹protein) at pH 9 and 65° C. At 80° C. no activity could be measured. The apparent reaction is:
Methane+NAD⁺+H2O→methanol+NADH+H⁺

The Gibbs free energy change of this reaction is about +90 kJ per mol. Therefore, this reaction can only occur when the concentration of the substrates are high and the concentration of the products low. We could show that the reaction is inhibited by addition of methanol to the assay mixture and that a higher activity was measured when the NAD⁺ concentration was increased.

### Example 3

### Sulphate Reduction by Coculture with Thermotoga Species

Coculture experiments were performed by growing Archaeoglobus profundus with T. maritima at 80° C. and Desulfotomaculum sp. strain WW1 with T. lettingae at 65° C. (Table 2). In both cases, sulphate was utlised as the electron acceptor. The ratio of CO₂ formation from methane and sulphate conversion to sulphide were nearly 1:1 for both cocultures. The calculated rates of methane conversion were slower than in the case with thiosulphate, which was about 20 μmol per vial per day. Conversion of methane coupled to sulphate reduction led to more than ten-fold increased cell numbers of both the Thermotoga species and the sulphate-reducing microorganisms. The ¹³ C-carbon recoveries for the cocultures of T. maritima and T. lettingae were calculated to be 85% and 78%, respectively,

TABLE 2a ¹³C-methane oxidation by T. maritima (T.m.) in
coculture with Archaeoglobus profundus (A.p.) in the presence
of sulphate‡.

| | | | | Total | Number of | |
|---|---|---|---|---|---|---|
| | | Total | Total | Sulphide | cells | |
| | ¹³CH₄ | Sulphate | CO₂* | # | (ml)† | |
| Day 0 | 1.75 | 1.10 | 4.87 | 0.11 | 3 × 10⁵ | (T.m.) |
| | | | | | 2 × 10⁶ | (A.p.) |
| Day 40 | 1.07 | 0.31 | 5.53 | 0.85 | 5 × 10⁷ | (T.m.) |
| | | | | | 7 × 10⁸ | (A.p.) |
| Utilised/ | 0.68 | 0.79 | 0.57 | 0.74 | | |
| produced | | | | | | |

TABLE 2b ¹³C-methane oxidation by T. lettingae (T.l.) in
coculture with Desulfotomaculum sp. strain WW1 (WW1) in the
presence of sulphate‡.

| | | | | Total | Number of | |
|---|---|---|---|---|---|---|
| | | Total | Total | Sulphide | cells | |
| | ¹³CH₄ | Sulphate | CO₂* | # | (ml)† | |
| Day 0 | 1.75 | 1.10 | 4.87 | 0.07 | 3 × 10⁶ | (T.l.) |
| | | | | | 3 × 10⁶ | (WW1) |
| Day 40 | 1.21 | 0.50 | 5.28 | 0.53 | 2 × 10⁷ | (T.l.) |
| | | | | | 8 × 10⁸ | (WW1) |
| Utilised/ | 0.54 | 0.60 | 0.41 | 0.51 | | |
| produced | | | | | | |

For symbols see TABLE 1.

Previous results showed that T. maritima and T. lettingae are able to grow on other C₁-compounds like H₂—CO₂, formate, methanol, and methylamine in the presence thiosulphate and yeast extract¹⁸. We also observed that addition of 0.5 to 5.0 g/l of yeast extract resulted in better growth and consequently slightly faster methane oxidation than in the original medium. In the absence of yeast extract, the pure cultures could not grow, even not on glucose. The rate of methane utilisation could be increased from 32 to 33 μmol per vial per day for T. maritima and from 30 to 31 μmol per vial for T. lettingae when 2.5 g/l yeast extract was added in the medium. However, similarly to the findings within original medium which contained 0.15 g/l of yeast extract, methane oxidation started only after around 10 days of incubation. When more than 5.0 g/l of yeast extract was added, growth for both organisms was better but methane oxidation rates were not higher than the obtained values.

1. A process for converting methane to produce hydrogen or hydrogen equivalents, characterised in that methane is subjected anaerobically to the activity of methane-oxidising bacteria of the order of the Thermotogales.

2. A process according to claim 1, wherein the methane-oxidising bacteria comprise a Thermotoga species.

3. A process according to claim 2, wherein the Thermotoga species comprises T. maritima or T. lettingae.

4. A process according to any one of claims 1-3, which is carried out at a temperature between 25 and 90° C.

5. A process according to any one of claims 1-4, which is carried out in the presence of thiosulphate.

6. A process for reducing chemical compounds by biological reduction using hydrogen equivalents, characterised in that the hydrogen equivalents are produced by subjecting methane to anaerobic methane-oxidising bacteria of the order of the Thermotogales.

7. A process according to claim 6, wherein sulphur compounds are reduced to sulphide using a sulphate-reducing species.

8. A process according to claim 7, wherein the sulphur compounds comprise sulphate and/or sulphite.

9. A process according to claim 7 or 8, wherein the anaerobic methane-oxidising species comprises a Thermotoga, Thermosipho or Fervidobacterium species.

10. A process according to claim 7 or 8, wherein the sulphate-reducing species comprises an Archaeglobus, Desulfotomaculum, Desulforomonas, Desulfovibrio or Thermodesulfovibrio species.

11. A process according to claim 6, wherein metals are reduced from a high valence state to a low-valence or zero-valence state.

12. A process according to any one of claims 6-11, wherein a temperature of between 25 and 90° C. is used.

13. A mixed culture, containing one ore more anaerobic methane-oxidising Thermotogales species, and one or more sulphate-reducing or metal-reducing Archaeglobus, Desulfotomaculum, Desulforomonas or Desulfovibrio species.
